(19) United States
(12) Patent Application Publication
(10) Pub. No.: US 2004/0253311 A1
(43) Pub. Date: Dec. 16, 2004

(54) Multi-layer tablet comprising non-steroidal anti-inflammatory drugs, decongestants and non-sedating antihist amines

(76) Inventors: Roger Berlin, Mendham, NJ (US); Peter Ramsey, Midlothian, VA (US); Arunya Usayapant, Ivanhoe, IL (US); Josephine Fubara, Richmond, VA (US); Scott Poxon, Mechanicsville, VA (US); Bill Bubnis, Mechanicsville, VA (US); David Giamalva, Glen Allen, VA (US); John Strode, Mechanicsville, VA (US); John Bachert, Richmond, VA (US); Michael Williams, Midlothian, VA (US)

Correspondence Address: HUNTON & WILLIAMS LLP, Intellectual Property Department, Riverfront Plaza, East Tower, 951 E. Byrd St., Richmond, VA, 23219-4074, US

(21) Appl. No.: 10/859,256

(22) Filed: Jun. 2, 2004

Related U.S. Application Data
(63) Continuation-in-part of application No. 10/740,386, filed on Dec. 17, 2003.
(60) Provisional application No. 60/434,342, filed on Dec. 18, 2002.

(51) Int. Cl. A61K 9/24; A61K 31/55; A61K 31/495; A61K 31/192

(52) U.S. Cl. 424/472; 514/569; 514/570; 514/217.05; 514/255.04; 514/649

(57) ABSTRACT

The present invention is directed to a pharmaceutical composition and method for treatment of rhinitis and cold like symptoms, the composition includes a non-steroidal anti-inflammatory drug (NSAID), a decongestant and an antihistamine preferably in a multi-layer tablet comprising an immediate release layer and an extended release layer to optimize the delivery profile and match the duration of biological action of the multiple active agents.

This application is a continuation-in-part Application of U.S. patent application Ser. No. 10/740386 filed Dec. 17, 2003 which claims the benefit of U.S. Provisional Application 60/434,342 filed Dec. 18, 2002, both of which are incorporated herein by reference.

### FIELD OF THE INVENTION

The present invention relates to improved dosage forms of pharmaceuticals for treating rhinitis associated with allergies and colds.

### BACKGROUND OF THE INVENTION

Decongestants and Antihistamines

Rhinitis refers to an inflammatory disorder of the nasal passages. The symptoms of rhinitis typically consist of sneezing, rhinorrhea, nasal congestion, and increased nasal secretions. Untreated rhinitis may lead to other disorders including infection of the sinuses, ears and lower respiratory tract.

Two types of oral medication are commonly used to treat rhinitis: decongestants and antihistamines. Decongestants and antihistamines differ in mechanism of action, therapeutic effects, and side effects. It is common practice to combine the use of decongestants and antihistamines to bring about more complete symptom relief of rhinitis than is possible with either entity alone.

Decongestants commonly used to treat rhinitis include the sympatomimetic agents pseudoephedrine and phenylephrine. These agents act to constrict vessels in the nasal mucus membranes and thereby decrease tissue swelling and nasal congestion. Decongestants are found to be better than antihistamines for restoring the patency of congested nasal airways. Nasal decongestants are stimulatory. Decongestants, however may produce nervousness, restlessness and insomnia, especially if taken at night.

Histamine is a mediator released from cells which line the walls of the nasal mucous membranes (mast cells). When released, histamine is known to bind to local receptors and thereby cause sneezing, nasal itching, swelling of the nasal membranes, and increased nasal secretions. Antihistamines relieve these effects, albeit by a different mechanism than decongestants. Antihistamines block the binding of histamine to histamine receptors in the nasal membranes. Side effects of antihistamines frequently include impairment of mental acuity and sedation.

Combinations of decongestants and antihistamines employ two mechanistic approaches, and have been shown to offer more complete relief of rhinitis symptoms than therapy with either component alone. Currently, many cold and allergy relief products contain both. Incorporation of decongestant and sedating antihistamine into a single dosage unit balances stimulation and sedation of the components. However, some individuals vary in their sensitivity to either the decongestant or the antihistamine. Consequently, some individuals experience an irritability and/or sedation with these combinations.

Examples of commercial formulations containing decongestant and sedating antihistamine include:

1. CHLOR-TRIMETON™ 4 hour Allergy/Decongestant which contains 4 mg of chlorpheniramine (sedating antihistamine) and 60 mg pseudoephedrine sulfate (stimulating decongestant), and which is recommended to be taken every 4 to 6 hours (½ this dosage for children 6 to under 12);

2. CHLOR-TRIMETON™ 12 hour Allergy/Decongestant which contains 8 mg of chlorpheniramine (sedating antihistamine) and 120 mg pseudoephedrine sulfate (stimulating decongestant), and which is recommended to be taken every 12 hours (adults and children over 12 years of age only);

3. BROMFED™ Tablets which contains 4 mg of brompheniramine (sedating antihistamine) and 60 mg pseudoephedrine sulfate (stimulating decongestant), and which is recommended to be taken every 4 to 6 hours (½ this dosage for children 6 to under 12);

4. BROMFED™ Capsules which contains 12 mg of brompheniramine (sedating antihistamine) and 120 mg pseudoephedrine sulfate (stimulating decongestant), and which is recommended to be taken every 12 hours (adults and children over 12 years of age only);

5. BENADRYL™ Allergy Decongestant Tablets which contains 25 mg of diphenhydramine hydrochloride (sedating antihistamine) and 60 mg pseudoephedrine sulfate (stimulating decongestant), and which is recommended to be taken by adults and children over 12 years of age every 4 to 6 hours, not to exceed 4 tablets in 24 hours; and

6. TAVIST-D™ Tablets which contains 1.34 mg clemastine fumarate (sedating antihistamine) and 75 mg phenylpropanolamine hydrochloride (stimulating decongestant), and which is recommended to be taken every 12 hours (adults and children over 12 years of age only).

Formulations have been commercialized that incorporate both a decongestant and a non-sedating antihistamine into a single dosage unit. While such formulations offer the advantage in being non-sedating, their efficacy does not approach that offered by sedating antihistamines, especially for rhinitis due to colds.

Non-Steroidal Anti-Inflammatory Drugs

Non-steroidal anti-inflammatory drugs (NSAIDS) are ideally suited for use in cold formulations for their analgesic, anti-inflammatory, and antipyretic activity and low incidence of side effects. Exemplary cold formulations containing non-steroidal anti-inflammatory agents include Advil Cold and Sinus™, Motrin Cold and Flu™, Motrin lB Sinus™ and Dristan Sinus™, each containing 200 mg ibuprofen and 30 mg pseudoephedrine.

U.S. Pat. No. 5,025,019 teaches pharmaceutical compositions and methods of using a composition containing a non-steroidal anti-inflammatory drug in combination with at least one other active component selected from an antihistamine, decongestant, cough suppressant or expectorant.

While these combination products provide effective symptom treatment of rhinitis due to cold and allergy, they do not alleviate the side effects of decongestants and antihistamines in sensitive individuals. Further these combination products do not provide extended non-drowsy relief for treating the range of rhinitis symptoms associated with allergies and /or a cold. Thus, there remains a need in the art for improved compositions and methods for treating symptoms of rhinitis with reduced side effects from the treatment and/or with improved release characteristics of the pharmaceutical actives.

### SUMMARY OF THE INVENTION

The present invention is directed to a pharmaceutical composition which includes an effective amount of each of a non-steroidal anti-inflammatory drug (NSAID), a decongestant, and an antihistamine.

In one embodiment the pharmaceutical composition comprises an effective amount of each of (a) a non-steroidal anti-inflammatory drug (NSAID), (b) a decongestant; and (c) an antihistamine, wherein the composition is a multi-layer tablet; and at least one of the NSAID, decongestant and antihistamine is in a first layer of the multi-layer tablet and at least one other of the NSAID, decongestant and antihistamine is in a second layer of the multi-layer tablet. Further the NSAID, decongestant and antihistamine may have substantially matched release profiles. At least one of the NSAID, decongestant, and antihistamine may be modified to extend the release profile.

NSAIDs suitable for use in the practice of the invention include, but are not limited to, a propionic acid derivatives, acidic acid derivatives, fenamic acid derivatives, biphenylcarboxylic acid derivatives, oxicams and cox-2 inhibitors. Decongestants suitable for use in the practice of the invention include, but are not limited to, pseudoephedrine, phenylephedrine and phenylpropanolamine. Antihistamines suitable for use in the practice of the invention include, but are not limited to, astemizole, azatadine, azelastine, acrivastine, brompheniramine, chlorpheniramine, clemastine, cyclizine, carebastine, cyproheptadine, carbinoxamine, descarboethoxyloratadine (also known as SCH-34117), desloratadine doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, picumast, pyrilamine, promethazine, terfenadine, tripelennamine, temelastine, trimeprazine and triprolidine.

Optionally the composition may further include an anti-tussive. Suitable anti-tussives include, but are not limited to, dextromethorphan, codeine and pholcodine.

In another embodiment a pharmaceutical composition comprising an effective amount of each of (a) a non-steroidal anti-inflammatory drug (NSAID); (b) a decongestant; and (c) an antihistamine wherein the composition is bi-layer tablet, and at least one of the NSAID, decongestant and antihistamine is in a first layer of the bi-layer tablet and at least one other of the NSAID, decongestant and antihistamine is in a second layer of the bi-layer tablet. Further the NSAID, decongestant and antihistamine may have substantially matched release profiles and at least one of the NSAID, decongestant and an antihistamine may be modified to extend the release profile.

In another embodiment pharmaceutical composition comprises naproxen, loratadine and pseudoephedrine wherein the naproxen, loratadine and pseudolphedrine are in a bi-layer tablet and wherein the naproxen and loratadine are in a first layer of the bi-layer tablet and pseudoephedrine suitably modified to extend the release profile to substantially match the release profiles of naproxen and loratadine is in a second layer of the bi-layer tablet.

The invention further comprises a method of relieving symptoms of rhinitis in a mammal, which includes administering: an antihistaminic effective amount of antihistamine, a decongestive effective amount of a decongestant, and an anti-inflammatory effective amount of a non-steroidal anti-inflammatory agent (NSAID), wherein the antihistamine, decongestant and NSAID are in a multi-layer tablet and at least one of NSAID, decongestant and antihistamine is in a first layer of the multi-layer tablet and at least one other of the antihistamine, decongestant and NSAID is in a second layer of the multi-layer tablet and wherein the release profiles of the NSAID, decongestant and antihistamine are substantially matched.

The invention further comprises a method of preparing a pharmaceutical composition, the method including selecting a non-steroidal anti-inflammatory drug (NSAID), a decongestant, and an antihistamine, determining the duration of action profile of each of the NSAID, decongestant and antihistamine, preparing at least one immediate release matrix comprising at least one of the NSAID, decongestant and antihistamine, preparing at least one extended release matrix comprising at least one of the NSAID, decongestant and antihistamine and wherein the at least one NSAID, decongestant and antihistamine of the extended release matrix has an extended release profile, and forming the at least one immediate release matrix and the at least one extended release matrix into a layered solid dosage form.

The solid dosage form of the invention is preferably a multi-layer tablet. The multi-layer tablet may be formed by compression of the immediate release matrix and the extended release matrix, by coating one of the immediate release and extended release matrices on the other of the immediate release and extended release matrices, or by creating a tablet within a tablet wherein in one portion is formed from the extended release matrix and the other portion is formed from the immediate release matrix.

The invention further includes a pharmaceutical composition comprising an active pharmaceutical agent and an amount of magnesium oxide wherein the amount of magnesium oxide is sufficient to modify the release profile of the active pharmaceutical agent and a method for preparing the composition.

### BRIEF DESCRIPTION OF FIGURES

FIG. 1 is a graph showing dissolution of pseudoephedrine HCl from MgO-HPMC matrices.

### DETAILED DESCRIPTION OF THE INVENTION

In application U.S. Ser. No. 10/740 386, to which the present application claims priority, the inventors disclosed their surprising finding that a pharmaceutical composition comprising a non-steroidal anti-inflammatory agent (NSAID), a decongestant and an antihistamine in which either the antihistamine or decongestant or both were present in an amount less than about 75% of the present approved dosage for the decongestant or the antihistamine and in which the NSAID was present in about 100% of the normal dosage strength provided effective relief of rhinitis symptoms. The compositions of the present invention may use either the dosages discussed and claimed in application U.S. Ser. No. 10/740 386 or conventional dosage amounts.

In the present invention it has been discovered that a combination of a non-steroidal anti-inflammatory agent, a decongestant, and an antihistamine (preferably a non-sedating antihistamine) can be prepared in a solid dosage form, preferably a multi-layer tablet comprising an immediate release layer and an extended release layer, to provide a composition with an optimized delivery profile and a matched duration of action of the active agents such that their durations of effect are substantially equal. Matched duration of action provides for relief of the range of symptoms for a specified length of time and alleviates the need for taking multiple dosages of various symptom specific drugs on different schedules to maintain relief from the range of rhinitis symptoms. In addition to convenience, this reduces the potential for a patient to take an inappropriate amount or combination of drugs.

“Matched release profile” as used herein means that the duration of action of each of pharmaceutical actives in the pharmaceutical composition as presented to the patient is substantially equal. This may be accomplished by selecting active agents known to have similar durations of action or by modification of one or more of the active agents to alter the duration of action. The modification may mean modification of the pharmaceutical active per se or modification via the of a matrix (e.g. excipients) used with the pharmaceutical active. Methods of determination of the duration of action or biological profile of a pharmaceutical active are known to those skilled in the art.

The term “extended release matrix” as used herein means a composition comprising at least one pharmaceutical active and excipients wherein either modifications to the pharmaceutical active per se or modifications of the pharmaceutical active as a result of combination with the excipients yields a composition that when administered to a mammal gives a duration of action substantially longer than the unmodified pharmaceutical active.

The term “immediate release matrix” means a composition comprising at least one pharmaceutical active and excipients in which there is no substantial alteration of the release profile of the pharmaceutical active.

In one embodiment the composition of the invention comprises a bi-layer tablet, including an immediate release layer comprising loratadine and naproxen sodium which have comparable biological half-lives which are sufficient to provide a 12-24 hour duration of action without further modification. Not only do naproxen sodium and loratadine have comparable biological half-lives, but also the inventors have surprisingly discovered that the combination of naproxen sodium and loratadine dissolves faster in aqueous media than each separately. In this embodiment, pseudoephedrine HCl, modified to extend the release profile such that the profile is comparable to that of naproxen and loratadine, is used in the extended release layer. The compositions of the invention provide an improved dosage in which extended relief from the range of symptoms manifested by rhinitis is provided in a dosage unit that is preferably taken at 12 or 24 hour intervals.

As used herein, the term “rhinitis” refers to inflammation of the nasal mucous membranes, which could result from a cold, flu, or allergies. Rhinitis may be characterized by one or more cold-like symptoms.

Cold-like symptoms as used herein refers to coryzea, nasal congestion, upper respiratory infections, allergic rhinitis, otitis, sinusitis, and the like. Runny nose and nasal congestion can also be cold symptoms.

The terms “effective amount” or “therapeutically effective amount” of an active agent as provided herein is defined as an amount of the agent at least sufficient to provide the desired therapeutic effect.

The term “normal approved dose” or “approved dose” of an active agent as provided herein is defined as an amount of the active agent that has been approved as safe and effective by the United States Food and Drug Administration for administration in humans in a particular dosage form. An approved dose is thus a dose found in a pharmaceutical product, an amount of active agent per unit dosage form. In the present invention, reference to a ratio of approved doses means doses approved for the same patient population (e.g., adult to adult or pediatric to pediatric), -and approved for the same dosage form (e.g., elixir, tablet, capsule, caplet, controlled release, etc.).

The term “active pharmaceutical agent,” “pharmaceutical active,” “active agent” and “drug” as used herein should be considered to have the same meaning.

In the practice of the invention, the improved solid dosage forms of the composition of the invention comprises three different drugs from the three different classes of action: non-steroidal anti-inflammatory drugs (NSAIDs), decongestants and antihistamines (preferably non-sedating antihistamines). Selection of a drug from each of these classes is desirable to address the range of symptoms manifested by rhinitis associated with allergies and colds. These symptoms included sneezing, runny nose, itchy watery eyes, itchy nose and throat, sinusitis, nasal congestion, sinus pressure, minor aches, pains and headaches. It is desirable that the drugs selected either be similar in duration of action or that they be modified to have a similar duration of action.

Modifying the duration of action of one pharmaceutical active in the presence of a different pharmaceutical active which is preferentially unmodified can be problematic. The inventors have found that use of a multi-layer tablet facilitates accommodating use of release modifying agents with one or more active agents while minimizing impact of release modifying agents on active agents in other layers of the multi-layer tablet. The use of two or more layers in the tablet also allows for optimization of the matrix for each of the three drug components. Further, in some embodiments separation of active agents into two or more layers may facilitate reduction of undesirable interactions between components. In a preferred embodiment comprising loratadine, naproxen sodium and pseudoephedrine, loratadine and naproxen sodium have a suitable release profile unmodified, while the duration of action of unmodified pseudoephedrine is much shorter than that of loratadine and naproxen sodium. Accordingly, the unmodified loratadine and naproxen sodium may be combined in an immediate release layer while pseudoephedrine may be modified to extend release and included in a second layer of the tablet.

Methods known to those skilled in the art, including either dry or wet granulation methods, may be used to prepare the matrix of each layer of the multi-layer tablet. The thus prepared matrices may be used to form the layers of the multi-layer tablet. The layers may be formed from the matrices using direct compression processes, coating methods, and/or by the so called “tablet with-in a tablet” method. The use of the term “multi-layer tablet” is taken to mean a tablet with two or more layers of distinct compositions. The multi-layer tablet may be formed by any method that yields such a result, including compression using a two or three layer press or through compression using a tablet within a tablet press.

Antihistamines

The term “antihistamine”, as used in connection with treating nasal symptoms associated with allergy or cold, generally refers to histamine H₁ receptor antagonists. Numerous chemical substances are known to have histamine H₁ receptor antagonist activity. Many useful compounds can be classified as ethanolamines, ethylenediamines, alkylamines, phenothiazines or piperidines. Representative H₁ receptor antagonists, include, without limitation: astemizole, azatadine, azelastine, acrivastine, brompheniramine, chlorpheniramine, clemastine, cyclizine, carebastine, cyproheptadine, carbinoxamine, descarboethoxyloratadine (also known as SCH-34117), desloratadine doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, picumast, pyrilamine, promethazine, terfenadine, tripelennamine, temelastine, trimeprazine and triprolidine. Other compounds can readily be evaluated to determine activity at H₁ receptors by known methods, including specific blockade of the contractile response to histamine of isolated guinea pig ileum.

Of the foregoing histamine H₁ receptor antagonists, chlorpheniramine and loratadine are specifically exemplified herein. These antihistamines are particularly suitable because of their relatively long biological half-lives, allowing them to be effective for 12 hours with a single immediate release dose. Loratadine offers the further advantage that it is non-sedating.

The usual adult dosage of loratadine is 5 mg orally every 12 hours or 10 mg every 24 hours (i.e., daily) with a maximum safe dose of 40 mg per day. The usual pediatric dose is 5 mg per day in children 2-5 years of age and 10 mg per day in older children (8-12 years of age). In accordance with some embodiments of the present invention, the usual adult dose may be reduced to about 2.5 mg to 3.75 mg every 12 hours, or to about 5 mg to 7.5 mg daily, up to a maximum dose of about 30 mg. Similarly, in some embodiments of the invention, the pediatric daily dosage is about 2.5 mg to 3.75 mg daily for children 2-5 years of age and 5 mg to 7.5 mg daily for children 8-12 years of age. In a further embodiment, the invention permits combining a pediatric dosage of loratadine with an adult dosage of an NSAID such as, but not limited to, naproxen or ibuprofen.

The usual adult dosage of chlorpheniramine is 4 mg orally every 4-6 hours as needed, up to a maximum of 24 mg per day. The usual pediatric dosage of chlorpheniramine is 2 mg orally every 4-6 hours, up to a maximum of 12 mg per day. The preferred salt is chlorpheniramine maleate. In accordance with some embodiments of the present invention, the usual adult dosage may be thus reduced to 3 mg, or further to 2 mg, orally every 4-6 hours as needed, up to a maximum of 12-18 mg per day. Similarly, in some embodiments of the invention, the pediatric dosage is 1.5 mg, or 1 mg, orally every 4-6 hours, up to a maximum of 6-9 mg per day. In a further embodiment, the invention permits combining a pediatric dosage of chlorpheniramine with an adult dosage of an NSAID such as, but not limited to, ibuprofen.

Decongestants

The decongestants for use in the pharmaceutical compositions and methods of use of the present invention include, but are not limited to, pseudoephedrine, phenylephedrine, and phenylpropanolamine. One of skill in the art would know of many other appropriate decongestants and their approved dosages.

A preferred decongestant is pseudoephedrine. The usual adult dose of pseudoephedrine is 60 mg every 4-6 hours, up to a maximum of 240 mg per day. The usual pediatric dose of pseudoephedrine is 15 mg every 6 hours, up to a maximum of 60 mg per day for ages 2-5 and 30 mg every 6 hours, up to a maximum of 120 mg per day for ages 6-12. Thus, in some embodiments of the practice of the present invention, the adult dose can be reduced to 45 or 30 mg every 4-6 hours, with a maximum of 120 to 180 mg per day, and the pediatric dose can be reduced to about 11 or 7.5 mg every 6 hours, up to a maximum of 30-45 mg per day. From the foregoing it is apparent that in some embodiments the invention contemplates administering a double or greater pediatric dose of decongestants with a normal adult dose of an NSAID to an adult.

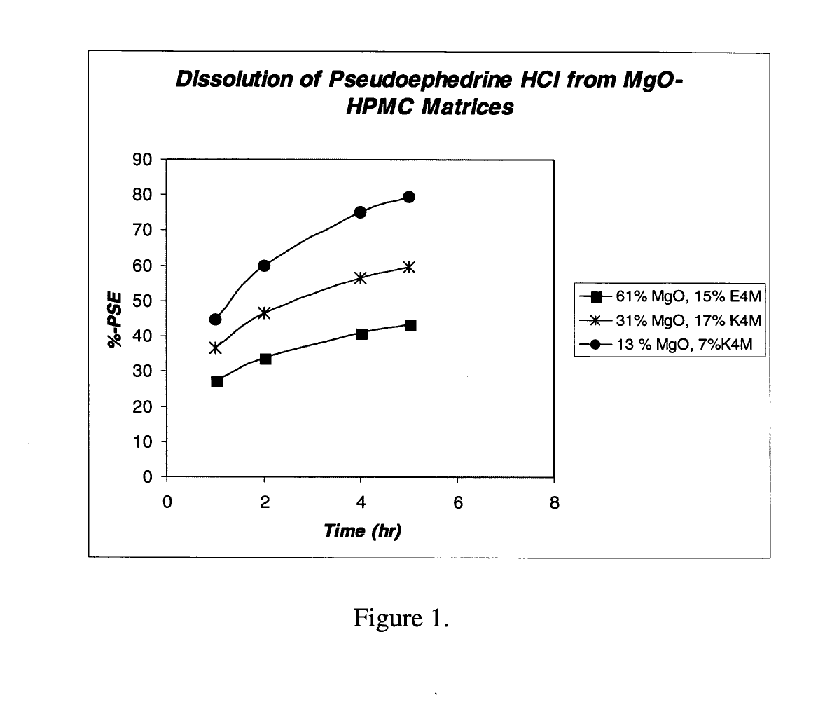

When modification of the release profile (e.g. duration of action) of the decongestant is desired, formulation approaches known to those skilled in the art may be used to modify the biological pharmacokinetic profile of the decongestant. These approaches may include, for example, the use of HPMC (hydroxypropylmethyl cellulose) to modify release of the active from a compacted tablet. Further, the inventors have surprisingly found that the use of HPMC in combination with magnesium oxide allows significant additional control of the release profile of the active active from a compacted tablet. For instance, magnesium oxide may be used to significantly decreased the release rate of commercially available pseudoephedrine from the extended release matrix prepared with HPMC. This approach may be used to minimize premature release of the active ingredient in an extended release matrix as shown in FIG. 1.

The use of magnesium oxide to modify the release of pseudoephedrine especially pseudoephedrine prepared with HPMC is exemplary. The inventions have found magnesium oxide to be generally useful for modulating the release profile for other pharmaceutical actives.

NSAIDS

The non-steroidal anti-inflammatory drugs (NSAIDs) for use in the pharmaceutical compositions and methods of use of the present invention may be selected from any of the following categories:

(1) The propionic acid derivatives;

(2) The acetic acid derivatives;

(3) The fenamic acid derivatives

(4) The biphenylcarboxylic acid derivatives;

(5) The oxicams, and

(6) Cox-2 inhibitors

Accordingly, the term “NSAID” as used herein is intended to mean any non-steroidal anti-inflammatory compound, including the pharmaceutically acceptable non-toxic salts thereof, falling within one of the six structural categories above.

The specific compounds falling within the foregoing definition of the non-steroidal anti-inflammatory drugs for use in the present invention are well known to those skilled in the art and reference may be found in various literature reference sources for their chemical structures, pharmacological activities, side effects, normal dosage ranges, etc. See, for example, Physician's Desk Reference and The Merck Index.

Of the propionic acid derivatives for use herein, ibuprofen, naxproxen, flurbiprofen, fenoprofen, ketoprofen, suprofen, fenbufen, and fluprofen may be mentioned as exemplified compounds. Of the acetic acid derivatives, exemplary compounds include tolmetin sodium, zomepirac, sulindac and indomethacin. Of the fenamic acid derivatives, exemplary compounds include mefenamic acid and meclofenamate sodium. Exemplary biphenylcarboxlic acid derivatives for use in the present invention include diflunisal and flufenisal. Exemplary oxicams include piroxicam, sudoxicam and isoxicam. Exemplary Cox-2 inhibitors include celecoxib, rofecoxib, meloxicam, and nimesulide. Of the foregoing non-steroidal anti-inflammatory drugs, in the practice of the exemplified embodiments of the present invention, ibuprofen and naproxen are preferred.

With respect to the dosage amount of the non-steroidal anti-inflammatory drugs in the compositions of the invention, although the specific dose will vary depending upon the age and weight of the patient, the severity of the symptoms, the incidence of side effects and the like, for humans, typical effective analgesic amounts of NSAIDs per dose are about 100-500 mg diflunisal, about 25-100 mg zomepirac sodium, about 50-400 mg ibuprofen, more preferably 100-200 mg ibuprofen, about 125-500 mg naproxen, about 25-100 mg flurbiprofen, about 50-199 mg fenoprogen, about 10-20 mg piroxicam, about 125-250 mg mefanaic acid, about 100-400 mg fenbufen or about 25-50 mg ketoprofen; however, greater or lesser amounts may be employed if desired or necessary.

In one embodiment of this invention naproxen, specifically its sodium salt, naproxen sodium is the NSAID. Naproxen is useful in part due to its long biological half-life (14±1 hours; Reference: Goodman & Gilman's The Pharmacological Basis of Therapeutics, 9^{th} Edition.). Optimally, naproxen sodium is used in combination with an antihistamine with a similar half-life such as chlorpheniramine or loratadine. The usual adult dosage of naproxen sodium is 220 mg orally every 12 hours as needed.

It was discovered in this invention that the combination of naproxen sodium and loratadine exhibits a synergistic effect on rates of in vitro dissolution of this combination. The solubility of naproxen is enhanced at a low pH in the presence of loratadine and the solubility of loratadine is enhanced at a high pH in the presence of naproxen. Further, in some instances the rate of dissolving appears to be enhanced for the combination as compared to the rates for naproxen and loratadine individually. This may be useful in that greater solubility is an advantage in immediate-release pharmaceutical preparations since the drug cannot be bio-available until it is dissolved.

The inventors anticipate, without wishing to be held to the theory, that similar synergistic solubility behavior would occur between loratadine and other pharmaceutically active carboxylic acids including, but not limited to, ibuprofen, ketoprofen, and their various salt forms; and between naproxen, ibuprofen, ketoprofen or their salts and amines such as chlorpheniramine, brompheniramine, diphenhydramine.

Anti-Tussitives

Optionally, anti-tussitives act on the brain to suppress the cough reflex. Such cough suppressants are used to relieve dry persistent coughs. The most commonly used drugs are dextromethorphan (an NMDA receptor antagonist), codeine and pholcodine (which are opioids). However, one skilled in the art would understand that there are many other well known and common anti-tussitives that may be used. The present invention is optionally directed to the use of anti-tussitves.

Pharmaceutical Compositions

Compositions of the invention are formulated in a single dosage form, and these may be solid (such as tablets, capsules, sachets, caplets, pellets, granules, trochets and the like), liquid (such as solutions or suspensions) or inhalation aerosols or patches. While the solid compounds will typically be administered orally, the liquids may be administered orally or by injection. Other dosage forms, such as suppositories, are also useful. The pharmaceutical composition described in the present invention may be formulated to release the active ingredients in a sustained release manner. Various formulations, including elixirs, suspensions, tablets, caplets, capsules, and the like are contemplated for dosage forms of these components.

A solid dosage form, consisting of a multi-layer tablet is the preferred dosage form for the compositions of this invention. For purposes of this specification and the accompanying claims, the term “tablet” refers equally to a tablet, a caplet or any other solid dosage form which is suitable for oral administration. Direct compression, dry granulation or wet granulation methods could be used for preparing the matrix of each layer. These methods are known to those skilled in the art.

In an exemplary embodiment, an immediate release matrix comprising loratadine and naproxen is combined in a bi-layer tablet with an extended release matrix comprising pseudoephedrine. This dosage form and composition is suitable for convenient once or twice a day oral administration as the release of pseudoephedrine is modified to match the duration of action of the immediate release matrix actives loratadine and naproxen. In one embodiment, a bi-layer tablet can be formulated for twice daily oral administration, consisting of an immediate release layer containing 5 mg loratadine and 220 mg naproxen sodium with appropriate excipients, and an extended release layer containing 120 mg pseudoephedrine hydrochloride with appropriate excipients.

In the exemplary embodiment comprising loratadine, naproxen and pseudoephedrine, the extended release matrix comprising pseudoephedrine is prepared by combining pseudoephedrine with suitable extended release polymers such as, but not limited to, hydroxypropyl methylcellulose and polyethylene oxide. It is preferable to use magnesium oxide in addition to the extended release polymer to further modify the release of pseudoephedrine. Other acceptable pharmaceutical excipients include, but are not limited to, fillers (such as lactose, mannitol, microcrystalline cellulose, magnesium oxide, and dibasic calcium phosphate) and glidants and/or lubricants (such as magnesium stearate, glycerol behenate, and silicon dioxide).

In the exemplary embodiment comprising loratadine, naproxen and pseudoephedrine, the immediate release matrix may comprise loratadine and naproxen sodium which are combined with various acceptable pharmaceutical excipients, including suitable fillers (such as dibasic calcium phosphate, lactose, microcrystalline cellulose, starch), suitable binders (such as PVP, HPMC, and HPC), suitable disintegrants (such as crospovidone and croscarmellose sodium), and suitable lubricants and/or glidants (such as glycerol behenate, talc, magnesium stearate, and silicon dioxide).

In another exemplary embodiment, a loratadine, ibuprofen and pseudoephedrine composition may be formulated in the dosage form of a bi-layer or tri-layer tablet suitable for convenient once or twice daily administration. In such an embodiment, it is desirable to modify the release profile of pseudoephedrine and ibuprofen of the dosage form to match the duration of action of the immediate release loratadine. In one embodiment, efficacious doses of ibuprofen and pseudoephedrine may both be included in an extended release matrix in the same layer of a bi-layer tablet. Alternatively, ibuprofen and pseudoephedrine may be included in separate extended release matrices of a tri-layer tablet. In either embodiment an efficacious dose of loratadine may be included in the immediate release layer of the tablet. Methods of modification of release profile such as those known to those skilled in the art may be used.

In another exemplary embodiment, a chlorpheniramine, naproxen and pseudoephedrine composition may be formulated in the dosage form of a bi-layer or tri-layer tablet suitable for convenient once or twice daily administration. In such an embodiment it is desirable to modify the release of pseudoephedrine and chlorpheniramine of the dosage form to match the duration of action of the immediate release naproxen. In one embodiment, efficacious doses of chlorpheniramine and pseudoephedrine may both be included in an extended release matrix in the same layer of a bi-layer tablet. Alternatively, chlorpheniramine and pseudoephedrine may be included in separate extended release matrices of a tri-layer tablet. In either embodiment, an efficacious dose of naproxen may be included in the immediate release layer of the tablet, for example.

Binders are agents used to impart cohesive qualities to the powdered material. Binders impart cohesiveness to the tablet formulation which insures the tablet remaining intact after compression, as well as improving the free-flowing qualities by the formulation of granules of desired hardness and size. Suitable binder materials include, but are not limited to, starch (including corn starch and pregelatinzed starch), gelatin, sugars (including sucrose, glucose, dextrose, lactose and sorbitol), polyethylene glycol, waxes, natural and synthetic gums, e.g., acacia, tragacanth, sodium alginate, celluloses, and Veegum, and synthetic polymers such as polymethacrylates and polyvinylpyrrolidone.

Lubricants have a number of functions in tablet manufacture. They prevent adhesion of the tablet material to the surface of the dies and punches, reduce interparticle friction, facilitate the ejection of the tablets from the die cavity and may improve the rate of flow of the tablet granulation. Examples of suitable lubricants include, but are not limited to, magnesium stearate, calcium stearate, stearic acid, glyceryl behenate, talc, sodium lauryl sulfate, sodium stearyl fumarate, polyethylene glycol or mixtures thereof. Generally, the lubricant is present in an amount from about 0.25% to about 5% of the weight of the final composition and more specifically from about 0.5 to about 1.5% of the weight of the final composition.

A disintegrant is a substance, or a mixture of substances, added to a tablet to facilitate its breakup or disintegration after administration. Materials serving as disintegrants have been classified chemically as starches, clay, celluloses, aligns, gums and cross-linked polymers. Examples of suitable disintegrants include, but are not limited to, croscarmellose sodium, sodium starch glycolate, starch, magnesium aluminum silicate, colloidal silicon dioxide, methylcellulose, agar, bentonite, alginic acid, guar gum, citrus pulp, carboxymethyl cellulose, microcrystalline cellulose, or mixtures thereof. Generally, the disintegrant is present in an amount from about 0.5% to about 25% of the weight of the final composition and more specifically from about 1% to about 15% of the weight of the final composition.

Glidants are substances which improve the flow characteristics of a powder mixture. Examples of glidants include, but are not limited to, colloidal silicon dioxide, talc or mixtures thereof. Generally, the glident is present in an amount of from about 0.1% to about 10% of the weight of the final composition and more specifically from 5 about 0.1% to about 5% of the weight of the final composition.

An adsorbent may be, for example, colloidal silicon dioxide, microcrystalline cellulose, calcium silicate or mixtures thereof. Generally, the adsorbent is present in an amount from about 0.05% to about 42% of the weight of the final composition and more specifically from about 0.05% to about 37% of the weight of the final composition.

If desired, other ingredients, such as diluents, stabilizers and anti-adherents, conventionally used for pharmaceutical formulations may be included in the present formulations. Optional ingredients include coloring and flavoring agents which are well known in the art.

The invention is further described by means of the following examples, which are not intended to limit the scope of the claimed invention in any manner.

### EXAMPLE 1

A bi-layer tablet was formulated for twice daily oral administration, comprising of an immediate release layer containing 5 mg loratadine and 220 mg naproxen sodium, and an extended release layer containing 120 mg pseudoephedrine hydrochloride.

The immediate release layer consisted of loratadine and naproxen sodium combined with various acceptable pharmaceutical excipients using a wet granulation method. Additional excipients were blended extra-granularly prior to compression. More specifically, the formula of the example comprised five intragranular ingredients: naproxen sodium, loratadine, povidone, talc, and croscarmellose sodium which were blended to form the immediate release wet granulation. Extragranular excipients were then blended into the immediate release granulation prior to tablet compression. Extragranular excipients included glyceryl behenate, silicon dioxide, croscarmellose sodium and microcrystalline cellulose.

Compression Blend for Immediate Release Layer

| Ingredient | % w/w | mg/finished layer |
|---|---|---|
| Immediate Release Wet Granulation | 72.23 | 252.81 |
| Naproxen Sodium | 87.00 | 220.00 |
| Loratadine | 2.00 | 5.10 |
| Povidone | 5.50 | 13.90 |
| Talc | 3.00 | 7.60 |
| Croscarmellose Sodium | 2.50 | 6.30 |
| Microcrystalline Cellulose | 21.27 | 74.44 |
| Croscarmellose Sodium | 2.50 | 8.75 |
| Silicon Dioxide | 2.00 | 7.00 |
| Glyceryl Behenate | 2.00 | 7.00 |
| Total layer weight | 100.00 | 350.00 |

For the extended release layer, pseudoephedrine HCl was combined with various acceptable pharmaceutical excipients using a wet granulation method, and additional excipients were blended extra-granularly prior to compression. More specifically, the example formula comprised of four intragranular ingredients: pseudoephedrine HCl, hydroxypropyl methylcellulose, mannitol and magnesium oxide which were blended to form the extended release granulation. Extragranular magnesium stearate was blended with the extended release granulation prior to tablet compression.

Compression Blend for Extended Release Layer

| Ingredient | % w/w | mg/finished layer |
|---|---|---|
| Extended Release Wet Granulation | 99.3 | 470.7 |
| Pseudoephedrine HCl | 25.5 | 120.0 |
| Hydroxypropyl Methylcellulose | 6.8 | 32.0 |
| Mannitol | 54.9 | 258.4 |
| Magnesium Oxide | 12.8 | 60.3 |
| Magnesium Stearate | 0.7 | 3.3 |
| Total layer weight | 100.0 | 474.0 |

The thus prepared granulations were compressed together to form the bi-layer tablet.

### EXAMPLE 2

Pharmaceutical compositions dosage forms for some embodiments of the present invention are made of the active ingredients listed below in the following dosage amounts.

TABLE 1

| NSAID | Decongestant | Antihistamine |
|---|---|---|
| Ibuprofen 200 mg | Pseudoephedrine 30 mg | Chlorpheniramine 2 mg |
| Ibuprofen 400 mg | Pseudoephedrine 60 mg | Chlorpheniramine 4 mg |
| Ibuprofen 200 mg | Pseudoephedrine 30 mg | Brompheniramine 2 mg |
| Ibuprofen 200 mg | Pseudoephedrine 30 mg | Diphenhydramine HCl 12.5 mg |
| Ibuprofen 200 mg | Phenylpropanolamine HCl 37.5 mg | Clemastine fumarate 0.67 mg |

### EXAMPLE 3

A study was run to evaluate and compare the analgesic/decongestant/antihistaminic efficacy of a caplet composition that includes an effective amount of each of ibuprofen, pseudoephedrine HCl and chlorpheniramine maleate, to a combination of the pseudoepedrine and chlorpheniramine in a tablet. The study was a multicenter, outpatient, multiple-dose, placebo controlled, double-blind, double-dummy, parallel-group, randomized trial.

Selection of Patients.

The study enrolled 1070 appropriately selected patients who were at least 12 years of age and included both males and females. Study participants were required to have: (1) at least a two year history of seasonal allergic rhinitis (which encompassed one or more of the following symptoms runny nose, itchy/watery/red eyes, nasal congestion, sneezing, itchy nose/throat/palate, allergy associated headache and facial pain/pressure/discomfort) and (2) a history of experiencing at least moderate headache, and/or facial pain/pressure/discomfort which worsened during the allergy season and responded to over-the-counter (OTC) analgesics (based upon self report). Qualified subjects were required to undergo a 3-30 day run-in phase to establish that the patients had sufficiently severe allergy symptoms, during which time they reflectively assessed the severity of the following symptoms in the morning (upon waking) and in the evening (prior to bedtime): nasal congestion, sneezing, rhinorrhea, itchy nose/throat/palate; itchy/watery/red eyes and headache/facial pain/pressure/discomfort. The severity of each symptom was assessed using a 4-point categorical scale where 0=none (no symptom present), 1=mild (minimal awareness of symptom, symptom easily tolerated), 2=moderate (symptom present and bothersome, but tolerable) and 3=severe (symptom hard to tolerate; may cause interference with daily activities/sleeping). They were also asked if the pain was a headache or facial pain/pressure/discomfort and were also asked to rank the pain from 0=none (no symptom present), 1=mild (minimal awareness of symptom, symptom easily tolerated), 2=moderate (symptom present and bothersome, but tolerable) and 3=severe (symptom hard to tolerate; may cause interference with daily activities/sleeping). Patients were administered the first dose of study medication (Day 1) when the patient experienced allergy-associated pain of at least moderate intensity and had a summed reflective allergy symptom score of at least 48 for the previous six morning and evening assessments of symptom severity. Patients were also assessed for allergy associated pain and were asked if they were in pain.

Of the 1070 subjects enrolled in the study and included in the safety analysis, 1044 qualified for the intent-to treat analysis and of these 1044 patients, 256 received Treatment 1; 265 received Treatment 2; 266 received Treatment 3; and 257 received Treatment 4. See below for a description of the treatments. Additionally 1032 patients were included in the modified intent to treat analysis.

Dosage Composition.

The patents in the study were randomly placed into four treatment groups. Treatment 1 consisted of one placebo tablet plus 2 ibuprofen/pseudoephedrine/chlorpheniramine (200/30/2 mg per caplet respectively) caplets for a total dose of 400/60/4 mg of ibuprofen/pseudoephedrine/chlorpheniramine every 6 hours. Treatment 2 consisted of one placebo tablet, one placebo caplet plus one ibuprofen/pseudoephedrine/chlorpheniramine (200/30/2 mg per caplet respectively) caplet every 6 hours. Treatment 3 consisted of one tablet of pseudoephedrine/chlorpheniramine (30/2 mg per tablet respectively) plus 2 placebo caplets every 6 hours. Treatment 4 consisted of 1 placebo tablet and two placebo caplets every 6 hours.

Dosage Timing and Monitoring of Symptoms.

The patients were dosed approximately every 6 hours 3 times per day (morning, midday and evening) up to a total of 19-21 doses over 7 days. Two and three hours after taking the first dose, the patients assessed the severity of their allergy-associated pain on a 4 point scale O=none (no symptom present), 1=mild (minimal awareness of symptom, symptom easily tolerated), 2=moderate (symptom present and bothersome, but tolerable) and 3=severe (symptom hard to tolerate; may cause interference with daily activities/sleeping). Prior to each subsequent dose the patients indicated whether or not they were experiencing any allergy-associated headache and/or facial pain/pressure/discomfort (yes or no answer required). On the evening of Day 1 (prior to bedtime) and on each morning (upon awakening) and evenings of Days 2-7 the patients self-assessed the severity of the following allergy symptoms: nasal congestion, sneezing, rhinorrhea, itchy nose/throat/palate, itchy/watery/red eyes, and allergy associated headache and/or facial pain/pressure/discomfort. The severity of each symptom was assessed using a 4-point categorical scale where O=none (no symptom present), 1=mild (minimal awareness of symptom, symptom easily tolerated), 2=moderate (symptom present and bothersome, but tolerable) and 3=severe (symptom hard to tolerate; may cause interference with daily activities/sleeping). On the evening of Day 7, after completing the allergy symptom assessment, subjects provided an overall assessment of the study medication, ranking the treatment for allergy symptoms on a scale of 0 (poor) to 4 (excellent). In addition, the patients evaluated any adverse experiences they experienced during the 7 day study medication period. The patients evaluated the adverse experience as either mild, moderate or severe. Examples of adverse experiences include, but are not limited to, somnolence, dry mouth, dizziness, and insomnia.

Criteria for Evaluation.

The primary efficacy parameter was the amount of change from the baseline in the 7 day, overall average reflective total symptoms score. Secondary Efficacy Parameters included key variables: (1) the time weighted sun of the pain intensity difference scores at two and three hours after the first dose of the study; (2) the change from the baseline in the overall average reflective total antihistamine symptoms score (sneezing, itchy/watery/red eyes, itchy nose/throat/palate) minus the overall average reflective total symptoms score; (3) the incidence of pre-dosing allergy associated pain (excluding the baseline measurement); (4) the change from the baseline in the average total reflective symptom score for each treatment day; (5) the overall evaluation of the study medication.

Statistical Analysis.

All analyses were performed using SAS® Version 6.12. Onset of symptom relief was defined as the first time point where a subject experienced a >15% reduction from baseline in the total symptom score. If a subject did not experience a >15% reduction from baseline during the entire course of the study, time to onset was censored and assigned a score of 7 days. Statistically significant treatment differences were declared if the probability of a random occurrence between the treatment groups was <0.05. Statistical trends were declared if the probability of random occurrence between treatment groups was 0.05<p<0.15 or the observed difference between treatment groups was at least 10%. Other appropriate statistical measures were employed as appropriate and as within the skill in the art to validate the study.

All variables based upon changes from baseline were analyzed via an ANOVA model including for effects of treatment, corresponding baseline and center. In addition, treatment-by-baseline and treatment-by-center interaction effects were assessed. The incidence of pre-dose allergy-associated pain (excluding baseline) was analyzed via a repeated measures logistics model. The model was fitted via generalized estimating equations with exchangeable correlation structure. Effects for treatment and baseline pain severity were included in the model. The overall evaluation of study medication scores was analyzed by the Cochran-Mantel-Haenszel test, controlling for center, using modified ridit scores. In addition, the treatment-by-center interaction was computed using the pseudo-homogeneity test. The distribution of time to onset of symptom relief was estimated using Kaplan-Meier estimates; the median time and their 95% confidence limits were derived by the method of Simon and Lee.

For each parameter analyzed via the ANOVA model, the 955 confidence interval for treatment differences was derived based upon on the differences in the least squares means and the corresponding standard error. The 95% confidence interval for each pair wise treatment difference in the incidence of allergy associated pain and the time to onset of symptom relief were based on the log-odds ratio and log hazard ratio, and their corresponding standard errors, respectively (based upon Wald's test). The confidence interval for treatment differences in the overall evaluation of the study medication was based on the Goodman-Kruskal Gamma statistic and its standard error.

Efficacy Results.

A total of 1044 patients were enrolled and took at least one dose study of medication. A total of 957 patients completed the study. A total of 1044 patients were included in the analysis of all efficacy variables, save for the time-weighted sum of the pain intensity difference, where 1032 patients were evaluated.

In the analysis of efficacy variables, the interaction effects of treatment-by-baseline and treatment-by-site were found to be generally not significant (p>0.1) across variables. The baseline means for individual reflective symptoms were nasal congestion 2.3, sneezing 1.7, runny nose 2.0, itchy nose/throat/palate 2.0, itchy/watery/red eyes 2.0 and headache/facial pain/pressure/discomfort 2.3, respectively. The mean reflexive symptom score and the mean total reflexive antihistamine score at baseline were 12.21 and 5.71, respectively. The allergy-associated pain when the first dose of the study medication was given to the patients was rated moderate by 49% and severe by 51% of the subjects among the 1032 patients who where evaluated for the time weighted sun? of the pain intensity difference. Almost all of the subjects (99.6%) were experiencing allergy associated pain at the time the first dose of study medication was taken. The treatment groups were comparable with respect to the above baseline variables as well as the baseline individual reflective symptoms.

The results of the primary efficacy parameter and key secondary parameters highlighted that the Treatment 1 and Treatment 2 (lower dosages) resulted in significantly better results than Treatment 3 (no ibuprofen) and Treatment 4 (full placebo).

The time-weighted sums of the pain intensity differences over 3 hours for the patients were 2.8 for the Treatment 1 and Treatment 2 groups. Treatment 3 and Treatment 4 had scores of 2.1 and 2.0, respectively. The mean change from baseline in the overall average reflective total symptom score for Treatments 1-4 were 5.6, 5.4, 4.6, and 3.8, respectively. The mean changes from baseline in the overall overage total reflective antihistamine symptoms score for Treatment groups were 2.9, 2.8, 2.4 and 1.9, respectively.

Thus the patients in the Treatment 2 group experienced a 33% improvement in the time-weighted sum of the pain intensity differences over 3 hours and a 17% improvement in the mean change from baseline in the overall average reflective total symptom score over the Treatment 3 group (no ibuprofen). The patients in Treatment 2 group also experienced a 47% improvement in the mean change from baseline in the overall overage total reflective antihistamine symptoms score over the Treatment 4 group. While the patients in Treatment 1 group exhibited numerically better composite scores for all of the allergy symptoms and histamine mediated symptoms, a statistical trend could not be established. In addition Treatment 1 and Treatment 2 groups had almost the same scores for the time-weighted sum of the pain intensity differences.

The results show that the 2 mg dose of chlorpheniramine is effective as an antihistamine, for it is more effective than the placebo in alleviating histamine-mediated symptoms. Also patients in Treatment group 1 identified an increase in somnolence as compared to Treatment group 2.

Conclusions.

The results of the study demonstrate that there is significant analgesic/decongestant/antihistaminic efficacy of ibuprofen/peudoephedrine/-chlorpheniramine at the given dosages in the treatment of the tested symptoms relative to decongestant and antihistamine alone. It was surprisingly found that ibuprofen contributes to the overall effectiveness of the combination by not only relieving allergy-associated pain but by also reducing the severity of other seasonal allergic rhinitis symptoms. This is shown by the overall greater effectiveness of Treatment 2 (which contains ibuprofen) compared to Treatment 3 (which did not contain ibuprofen). It also was surprisingly found that both dosages of ibuprofen/-pseudoephedrine/chlorpheniramine (400/60/4 and 200/30/2 mg total, respectively) were equally efficacious in relieving histamine-mediated symptoms of seasonal allergic rhinitis. In addition, a clear dose response was not seen between the Treatment 1 and Treatment 2 groups.

All treatments were tolerated and the incidence of adverse experiences were consistent with those reported for similar medications containing the same doses of pseudoephedrine. and chlorpheniramine. The proposed dose of ibuprofen/-pseudoephedrine/chlorpheniramine is 1-caplet every four to six hours—not to exceed 6 caplets in a 24-hour period—since both doses of I/P/C were equally effective and the 2-caplet I/P/C dose demonstrated an increased incidence of somnolence, dry mouth and asthenia relative to the 1-caplet dose. This dosing will allow for the product to be taken prior to bedtime and/or during the night (in addition to daytime dosing) and is consistent with the approved OTC daily dose of ibuprofen (1200 mg), and is still below the monograph daily doses of pseudoephedrine (240 mg) and chlorpheniramine (24 mg).

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

All patents, applications, articles, publications, and test methods mentioned above are hereby incorporated by reference.

What is claimed is:

1. A pharmaceutical composition comprising an effective amount of each of (a) a non-steroidal anti-inflammatory drug (NSAID), (b) a decongestant; and (c) an antihistamine, wherein the composition is a multi-layer tablet; and at least one of the NSAID, the decongestant and the antihistamine is in a first layer of the multi-layer tablet and at least one other of the NSAID, the decongestant and the antihistamine is in a second layer of the multi-layer tablet.

2. The pharmaceutical composition of claim 1, wherein the NSAID, the decongestant and the antihistamine have substantially matched release profiles.

3. The pharmaceutical composition of claims 1 or 2, wherein at least one of the NSAID, the decongestant, and the antihistamine has been modified to extend the release profile.

4. The composition of claim 1, wherein the NSAID comprises at least one of a propionic acid derivative, an acidic acid derivative, a fenamic acid derivative, a biphenylcarboxylic acid derivative, an oxicam and a cox-2 inhibitor.

5. The composition of claim 1, wherein the decongestant comprises at least one of pseudoephedrine, phenylephedrine and phenylpropanolamine.

6. The composition of claim 1, wherein the antihistamine comprises at least one of astemizole, azatadine, azelastine, acrivastine, brompheniramine, chlorpheniramine, clemastine, cyclizine, carebastine, cyproheptadine, carbinoxamine, descarboethoxyloratadine, desloratadine doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, picumast, pyrilamine, promethazine, terfenadine, tripelennamine, temelastine, trimeprazine and triprolidine.

7. The composition of claim 1, further comprising an anti-tussive.

8. The composition of claim 7 wherein the anti-tussive is at least one of dextromethorphan, codeine and pholcodine.

9. A pharmaceutical composition comprising an effective amount of each of (a) a non-steroidal anti-inflammatory drug (NSAID); (b) a decongestant; and (c) an antihistamine wherein the composition is bi-layer tablet, and at least one of the NSAID, the decongestant and the antihistamine is in a first layer of the bi-layer tablet and at least one other of the NSAID, the decongestant and the antihistamine is a second layer of the bi-layer tablet.

10. The pharmaceutical composition of claim 9, wherein the NSAID, the decongestant and the antihistamine have substantially matched release profiles.

11. The pharmaceutical composition of claims 9 or 10, wherein at least one of the NSAID, the decongestant and the antihistamine has been modified to extend the release profile.

12. The composition of claim 9, wherein the NSAID comprises at least one of a propionic acid derivative, an acidic acid derivative, a fenamic acid derivative, a biphenylcarboxylic acid derivative, an oxicam and a cox-2 inhibitor.

13. The composition of claim 9, wherein the decongestant comprises at least one of pseudoephedrine, phenylephedrine and phenylpropanolamine.

14. The composition of claim 9, wherein the antihistamine comprises at least one of astemizole, azatadine, azelastine, acrivastine, brompheniramine, chlorpheniramine, clemastine, cyclizine, carebastine, cyproheptadine, carbinoxamine, descarboethoxyloratadine, desloratadine doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, picumast, pyrilamine, promethazine, terfenadine, tripelennamine, temelastine, trimeprazine and triprolidine.

15. A pharmaceutical composition comprising naproxen, loratadine and pseudoephedrine wherein the naproxen, loratadine and pseudolphedrine are in a bi-layer tablet and wherein the naproxen and loratadine are in a first layer of the bi-layer tablet and the pseudoephedrine suitably modified to extend the release profile to substantially match the release profile of naproxen and loratadine is in a second layer of the bi-layer tablet.

16. A method of relieving symptoms of rhinitis in a mammal, which comprises administering:
(a) an antihistaminic effective antihistamine;
(b) a decongestive effective amount of a decongestant; and
(c) an anti-inflammatory effective amount of a non-steroidal anti-inflammatory agent (NSAID);
wherein the antihistamine, the decongestant and the NSAID are in a multi-layer tablet and at least one of the NSAID, the decongestant and the antihistamine is in a first layer of the multi-layer tablet and at least one other of the antihistamine, the decongestant and the NSAID is in a second layer of the multi-layer tablet and wherein the release profiles of the NSAID, the decongestant and the antihistamine are substantially matched.

17. A method of preparing a pharmaceutical composition, the method comprising:
(a) selecting a non-steroidal anti-inflammatory drug (NSAID), a decongestant, and an antihistamine;
(b) determining the duration profile of each of the NSAID, the decongestant and the antihistamine;
(c) preparing at least one immediate release matrix comprising at least one of the NSAID, the decongestant and the antihistamine;
(d) preparing at least one extended release matrix comprising at least one of the NSAID, the decongestant and the antihistamine and wherein the at least one of the NSAID, the decongestant and the antihistamine has been modified to extend the release profile; and
(e) forming the at least one immediate release matrix and the at least one extended release matrix into a layered solid dosage form.

18. The method of claim 17, wherein the solid dosage form is a multi-layer tablet.

19. The method of claim 17, wherein the multi-layer tablet is formed by compression of the immediate release matrix and the extended release matrix.

20. The method of claim 17, wherein the multi-layer tablet is formed by coating one of the immediate release matrix and the extended release matrix on the other of the immediate release matrix and the extend release matrix.

21. The method of claim 17, wherein the multi-layer tablet is formed by creating a tablet within a tablet wherein in a first tablet portion is formed from the extended release matrix and a second tablet portion is formed from the immediate release matrix.

22. A pharmaceutical composition comprising an active pharmaceutical agent and an amount of magnesium oxide wherein the amount of magnesium oxide is sufficient to modify the release profile of the active pharmaceutical agent.

23. The composition of claim 22 further comprising an extended release polymer.

24. The composition of claim 23 wherein the extended release polymer comprises at least one of hydroxypropyl methylcellulose and polyethylene oxide.

25. A method of preparing an extended release pharmaceutical composition comprising providing a pharmaceutical active and an amount of magnesium oxide wherein the amount of magnesium oxide is sufficient to modify the release profile of the active pharmaceutical agent.
